(19) United States
(12) Patent Application Publication
(10) Pub. No.: US 2022/0152025 A1
(43) Pub. Date: May 19, 2022

(54) INHALED IMATINIB FOR PULMONARY HYPERTENSION

(71) Applicants: United Therapeutics Corporation, Silver Spring, MD (US); MannKind Corporation, Westlake Village, CA (US)

(72) Inventors: Adam Marc Silverstein, Cary, NC (US); Patrick Poisson, Chapel Hill, NC (US); Ajay Keshava, Raleigh, NC (US); John J. Freeman,, JR., New Fairfield, CT (US); James Mills, Southbury, CT (US)

(73) Assignees: United Therapeutics Corporation, Silver Spring, MD (US); MannKind Corporation, Westlake Village, NY (US)

(21) Appl. No.: 17/528,011

(22) Filed: Nov. 16, 2021

Related U.S. Application Data
(60) Provisional application No. 63/114,781, filed on Nov. 17, 2020.

(51) Int. Cl. A61K 31/506; A61K 9/00

(52) U.S. Cl. CPC A61K 31/506; A61K 9/0075

(57) ABSTRACT

Methods of treatment of pulmonary hypertension comprising dry powder inhalation administration of imatinib, a pharmaceutically acceptable salt, or a derivative thereof, are provided. Dry powder inhalable compositions comprising imatinib, a pharmaceutically acceptable salt, or a derivative thereof, are also provided as well as methods of making the same.

The present application claimed priority to U.S. provisional application No. 63/114,781 filed Nov. 17, 2020, which is incorporated herein by references for all purposes.

### FIELD

The present application relates to methods, compositions, and kits for therapeutic treatment and, more particularly, to therapeutic methods involving administering imatinib using inhalation, such as dry powder inhalation, to treat pulmonary hypertension.

### BACKGROUND

All blood is driven through the lungs via the pulmonary circulation in order, among other things, to replenish the oxygen which it dispenses in its passage around the rest of the body via the systemic circulation. The flow through both circulations is in normal circumstances equal, but the resistance offered to it in the pulmonary circulation is generally much less than that of the systemic circulation. When the resistance to pulmonary blood flow increases, the pressure in the circulation is greater for any particular flow. The above described condition is referred to as pulmonary hypertension (PH). Generally, pulmonary hypertension is defined through observations of pressures above the normal range pertaining in the majority of people residing at the same altitude and engaged in similar activities.

Pulmonary hypertension may occur due to various reasons and the different entities of pulmonary hypertension were classified based on clinical and pathological grounds in 5 categories according to the latest WHO convention, see e.g. Simonneau G., et al. J. Am. Coll. Cardiol. 2004; 43(12 Suppl S):5S-12S. Pulmonary hypertension can be a manifestation of an obvious or explicable increase in resistance, such as obstruction to blood flow by pulmonary emboli, malfunction of the heart's valves or muscle in handling blood after its passage through the lungs, diminution in pulmonary vessel caliber as a reflex response to alveolar hypoxia due to lung diseases or high altitude, or a mismatch of vascular capacity and essential blood flow, such as shunting of blood in congenital abnormalities or surgical removal of lung tissue. In addition, certain infectious diseases, such as HIV and liver diseases with portal hypertension may cause pulmonary hypertension. Autoimmune disorders, such as collagen vascular diseases, also often lead to pulmonary vascular narrowing and contribute to a significant number of pulmonary hypertension patients. The cases of pulmonary hypertension remain where the cause of the increased resistance is as yet inexplicable are defined as idiopathic (primary) pulmonary hypertension (iPAH) and are diagnosed by and after exclusion of the causes of secondary pulmonary hypertension and are in the majority of cases related to a genetic mutation in the bone morphogenetic protein receptor-2 gene. The cases of idiopathic pulmonary arterial hypertension tend to comprise a recognizable entity of about 40% of patients cared for in large specialized pulmonary hypertension centers. Approximately 65% of the most commonly afflicted are female and young adults, though it has occurred in children and patients over 50. Life expectancy from the time of diagnosis is short without specific treatment, about 3 to 5 years, though occasional reports of spontaneous remission and longer survival are to be expected given the nature of the diagnostic process. Generally, however, disease progress is inexorable via syncope and right heart failure, and death is quite often sudden.

Pulmonary hypertension refers to a condition associated with an elevation of pulmonary arterial pressure (PAP) over normal levels. In humans, a typical mean PAP is approximately 12-15 mm Hg. Pulmonary hypertension, on the other hand, can be defined as mean PAP above 25 mm Hg, assessed by right heart catheter measurement. Pulmonary arterial pressure may reach systemic pressure levels or even exceed these in severe forms of pulmonary hypertension. When the PAP markedly increases due to pulmonary venous congestion, i.e., in left heart failure or valve dysfunction, plasma can escape from the capillaries into the lung interstitium and alveoli. Fluid buildup in the lung (pulmonary edema) can result, with an associated decrease in lung function that can in some cases be fatal. Pulmonary edema, however, is not a feature of even severe pulmonary hypertension due to pulmonary vascular changes in all other entities of this disease.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature, which result in a decreased cross-sectional area of the pulmonary blood vessels. This may be caused by, for example, chronic hypoxia, thromboembolism, collagen vascular diseases, pulmonary hypercirculation due to left-to-right shunt, HIV infection, portal hypertension, or a combination of genetic mutation and unknown causes as in idiopathic pulmonary arterial hypertension.

Pulmonary hypertension has been implicated in several life-threatening clinical conditions, such as adult respiratory distress syndrome (“ARDS”) and persistent pulmonary hypertension of the newborn (“PPHN”). Zapol et al., Acute Respiratory Failure, p. 241-273, Marcel Dekker, New York (1985); Peckham, J. Ped. 93:1005 (1978). PPHN, a disorder that primarily affects full-term infants, is characterized by elevated pulmonary vascular resistance, pulmonary arterial hypertension, and right-to-left shunting of blood through the patent ductus arteriosus and foramen ovale of the newborn's heart. Mortality rates range from 12-50%. Fox, Pediatrics 59:205 (1977); Dworetz, Pediatrics 84:1 (1989). Pulmonary hypertension may also ultimately result in a potentially fatal heart condition known as “cor pulmonale,” or pulmonary heart disease. Fishman, “Pulmonary Diseases and Disorders” 2^{nd} Ed., McGraw-Hill, New York (1988).

Imatinib functions as a specific inhibitor of a number of tyrosine kinase (TK) enzymes. It occupies the TK active site, leading to a decrease in activity. There are a large number of TK enzymes in the body, including the insulin receptor. Imatinib is specific for the TK domain in abl (the Abelson proto-oncogene), c-kit and PDGF-R (platelet-derived growth factor receptor). Aberrant expression and signaling of PDGF ligands and receptors is associated with several connective tissue disorders and lung diseases such as pulmonary arterial hypertension (PAH), lung cancer and idiopathic pulmonary fibrosis (IPF).

### SUMMARY

One embodiment is a method of treating pulmonary hypertension comprising administering, by inhalation, to a subject in need thereof, a therapeutically effective amount of a composition comprising a tyrosine kinase inhibitor, including, for example, imatinib, a pharmaceutically acceptable salt, or a derivative thereof.

Another embodiment is dry powder inhalable composition comprising a therapeutically effective amount of imatinib, a pharmaceutically acceptable salt, or a derivative thereof, and optionally one or more excipients. In an exemplary embodiment, the dry powder inhalable composition comprises crystalline particles of a diketopiperazine as a pharmaceutically acceptable excipient.

In one embodiment, there is provided an inhaler, such as a dry powder inhaler, for delivering an inhalable pharmaceutical composition, such as a dry powder composition, comprising a therapeutically effective amount of imatinib, a pharmaceutically acceptable salt, or derivative thereof, and optionally one or more excipients. In some embodiments, the dry powder inhaler can be structurally configured to deliver the dry powder composition from a capsule or a cartridge which can be adapted or mounted in the inhaler.

In one embodiment, the dry powder inhaler can be breath-actuated or activated to initiate powder aerosolization within the inhaler during use and delivery of the dry powder pharmaceutical composition.

### FIGURES

FIGS. 1A-1C show how the imatinib content of the powder affects the geometric particle size distribution of the powder discharged from the inhaler. The plots track three cut-points in the distribution: x₅₀ (median) (FIG. 1B) and points corresponding to approximately ±1 standard deviation from the median on a log-normal distribution (x₁₆ (FIG. 1A), and x₈₄ (FIG. 1C)).

FIG. 2 shows the estimated lung dose of imatinib as a function of imatinib content in crystalline carrier (XC) powders.

FIG. 3 shows concentrations of Imatinib in Rat Plasma Samples as a function of time.

FIG. 4 schematically illustrates identification of lung section.

FIG. 5 shows plots of concentrations of Imatinib in Rat Lung Sections identified in FIG. 4 as a function of time. Error Bars not Shown.

FIG. 6 shows concentrations of Imatinib in Rat Lung Sections identified in FIG. 4.

FIG. 7 shows concentrations of Imatinib in Rat Lung Sections identified in FIG. 4. Scaled for Visualizing the Lower Concentrations.

### DETAILED DESCRIPTION

A therapeutically effective dose of a tyrosine kinase inhibitor, including, for example, imatinib, gefitinib, erlotinib and sunitinib can be administered by inhalation using a an inhalation device, which may a compact inhalation device, such a dry powder inhaler, as an effective treatment for pulmonary hypertension. In some embodiments, imatinib is administered as a dry powder composition using a dry powder inhaler. Furthermore, such administering does not cause significant side effects, for example, pulmonary edema or subdural hematoma.

Methods of Treatment

Accordingly, one exemplary embodiment is a method of delivering to a subject suffering from pulmonary hypertension, such as a human being, a therapeutically effective amount of imatinib, a pharmaceutically acceptable salt, or a derivative thereof, comprising administering to the subject a composition comprising a therapeutically effective amount of imatinib, its derivative, or a pharmaceutically acceptable salt thereof by inhalation. In some embodiments, the composition is a dry powder inhalable composition that can be administered via a dry powder inhaler to a subject affected with a condition or disease, such as pulmonary hypertension, which can be treated by imatinib.

Another embodiment is a method for treating pulmonary hypertension, comprising administering to a subject in need thereof, such as a human being, imatinib, or a derivative thereof, or a pharmaceutically acceptable salt thereof, in a dry powder composition, using a dry powder inhaler.

Imatinib mesylate is also known as STI-571. The molecular weight of imatinib is 493.603, and its empirical formula is C₂₉H₃₁N₇O. Imatinib, or 4-[(4-methylpiperazin-1-yl)methyl]-N-[4-methyl-3-[(4-pyridin-3-ylpyrimidin-2-yl)amino]phenyl]benzamide, has the formula:

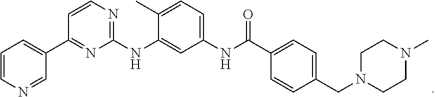 [SMILES: Cc1ccc(NC(=O)c2ccc(CN3CCN(C)CC3)cc2)cc1Nc1nccc(-c2cccnc2)n1]

Imatinib is a small molecule kinase inhibitor used to treat certain types of cancer. It is currently marketed by Novartis as Gleevec® (USA) or Glivec® (Europe/Australia) as its mesylate salt, imatinib mesilate (INN). It is used in treating chronic myelogenous leukemia (CML), gastrointestinal stromal tumors (GISTs) and a number of other malignancies.

Imatinib was first described in U.S. Pat. No. 5,521,184. US Patent Publication No. US20110190313A1 describes use of imatinib for treatment of pulmonary hypertension. US Patent Publication No. US20150044288A1 describes administration of imatinib by inhalation for treatment of pulmonary hypertension and other conditions.

The present disclosure also encompasses methods of using imatinib or its derivatives, or pharmaceutically acceptable salts thereof. In one embodiment, a method uses imatinib mesylate, currently marketed under the trade name of Gleevec®. The FDA has approved imatinib mesylate for the treatment of chronic myeloid leukemia (CML), gastrointestinal stromal tumors, relapsed or refractory Philadelphia chromosome-positive acute lymphoblastic leukemia (Ph+ALL), myelodysplastic/myeloproliferative diseases associated with platelet-derived growth factor receptor gene rearrangements, aggressive systemic mastocytosis without or an unknown D816V c-KIT mutation, hypereosinophilic syndrome and/or chronic eosinophilic leukemia who have the FIP1L1-PDGFRα fusion kinase (CHIC2 allele deletion) or FIP1L1-PDGFRα fusion kinase negative or unknown, unresectable, recurrent and/or metastatic dermatofibrosarcoma protuberans.

In certain embodiments, imatinib can be administered in combination with one or more additional active agents. In some embodiments, such one or more additional active agents can be also administered together with imatinib using a metered dose inhaler or a dry powder inhaler. Yet in some embodiments, such one or more additional active agents can be administered separately from imatinib. In some embodiments, the separate administration may be selected from oral, nasal, sublingual, buccal, intravenous, intramuscular, transdermal, liquid or gas aerosol inhalation (i.e., via metered dose or dry powder inhalers), rectal, or vaginal. Particular additional active agents that can be administered in combination with imatinib may depend on a particular disease or condition for treatment or prevention of which imatinib is administered, for example, pulmonary hypertension. In some cases, the additional active agent can be a calcium channel blocker, such as amlodipine; an endothelin receptor antagonist such as ambrisentan, bosentan, or macitentan; a phosphodiesterase type 5 inhibitor such as sildenafil or tadalafil; a prostacyclin analogue such as epoprostenol, iloprost, or treprostinil; or a prostacyclin IP receptor agonist such as selexipag.

In certain embodiments, the present disclosure extends to methods of using physiologically acceptable salts of imatinib, as well as non-physiologically acceptable salts of imatinib that may be used in the preparation of the pharmacologically active compounds.

The term “pharmaceutically acceptable salt” refers to a salt of imatinib with an inorganic base, organic base, inorganic acid, organic acid, or basic or acidic amino acid. In some embodiments, a salt of imatinib is a salt with an inorganic acid, organic acid, or acidic amino acid. In some embodiments, the counterion of the salt form of imatinib, is acetate, acetonide, alanine, aluminum, arginine, ascorbate, asparagine, aspartic acid, benzathine, benzoate, besylate, bisulfate, bisulfite, bitartrate, bromide, calcium, carbonate, camphorsulfonate, cetylpridinium, chloride, chlortheophyllinate, cholinate, citrate, cysteine, deoxycholate, diethanolamine, diethylamine, diphosphate, diproprionate, disalicylate, edetate, edisylate, estolate, ethylamine, ethylenediamine, ethandisulfonate, fumarate, gluceptate, gluconate, glucuronate, glutamic acid, glutamine, glycine, hippurate, histidine, hydrobromide, hydrochloride, hydroxide, iodide, isethionate, isoleucine, lactate, lactobionate, laurylsulfate, leucine, lysine, magnesium, malate, maleate, mandelate, meglumine, mesylate, metabisulfate, metabisulfite, methionine, methylbromide, methylsulfate, methyl p-hydroxybenzoate, mucate, naphthoate, napsylate, nitrate, nitrite, octadecanoate, oleate, ornithine, oxalate, pamoate, pentetate, phenylalanine, phosphate, piperazine, polygalacturonate, potassium, procaine, proline, propionate, propyl p-hydroxybenzoate, saccharin, salicylate, selenocysteine, serine, silver, sodium, sorbitan, stearate, succinate, sulfate, sulfite, sulfosalicylate, tartrate, threonine, tosylate, triethylamine, triethiodide, trifluoroacetate, trioleate, tromethamine, tryptophan, tyrosine, valerate, valine, xinafoate, or zinc. In some embodiments, a salt form of imatinib may be imanitib mesylate. In some embodiments, a salt of imatinib may be imatinib fumarate. In some embodiments, a salt of imanitib may be imatinib hydrochloride. In some embodiments, an imatinib salt may be imanitib phosphate.

In some embodiments, an imatinib salt may be in a crystalline form. For example, in some embodiments, imanitib mesylate may be in a crystalline form. In some embodiments, imatinib fumarate may be in a crystalline form. In some embodiments, imatinib hydrochloride may be in a crystalline form. In some embodiments, imanitib phosphate may be in a crystalline form.

In some embodiments, imatinib may be administered by an inhalation device, such as a pulsed inhalation device, which may contain a solution, a suspension or a powder comprising imatinib or its salt. For example, such solution or suspension may be used for aerosolizing or a nebulizing by an inhalation device, such as a nebulizer and/or a metered dose inhaler. Pulsed inhalation devices are disclosed, for example, in U.S. patent application publication No. 20080200449, U.S. Pat. Nos. 9,358,240; 9,339,507; 10,376,525; and 10,716,793, each of which is incorporated herein by reference in its entirety.

A metered dose inhaler in the present context means a device capable of delivering a metered or bolus dose of respiratory drug, such as imatinib, to the lungs. One example of the inhalation device can be a pressurized metered dose inhaler, a device which produces the aerosol clouds for inhalation from solutions, solids, and/or suspensions of respiratory drugs. In some embodiments, the aerosol clouds may be formed from a solution of a respiratory drug, such as imatinib or its salt, in chlorofluorocarbon (CFC) and/or hydrofluoroalkane (HFA).

In some embodiments, an inhalation device may be a single dose inhalation device, which may contain a unit dose container, such as a capsule or a cartridge with a single dose or multiple doses, e.g. 2 or more doses, of a respiratory drug, such as imatinib or its salt. In some embodiments, such device may be a dry powder inhaler.

In some embodiments, the inhalation device may be a dry powder inhaler. In some embodiments, the inhalation device, such as a pulsed inhalation device, may be a dry powder inhaler, which may contain a dry powder composition or formulation comprising imatinib or its salt, such imatinib mesylate. In some embodiments, in addition to imanitib or its salt, such as imatinib mesylate, the dry powder composition may further a diketopiperazine, such as (E)-3,6-bis[4-(N-carbonyl-2-propenyl)amidobutyl]-2,5-diketopiperazine (FDKP). In certain embodiments, the dry powder composition may consist of imanitib or its salt, such as imatinib mesylate, and diketopiperazine, such as (E)-3,6-bis[4-(N-carbonyl-2-propenyl)amidobutyl]-2,5-diketopiperazine (FDKP). Yet in certain embodiments, in addition to imanitib or its salt, such as imatinib mesylate, and diketopiperazine, such as (E)-3,6-bis[4-(N-carbonyl-2-propenyl)amidobutyl]-2,5-diketopiperazine (FDKP), the dry powder composition can further comprise pharmaceutically acceptable carriers, and/or excipients, for example, amino acids, for example, leucine, isoleucine, norleucine, methionine and glycine; sugars, including, trehalose, mannitol and lactose. In one embodiment, the dry powder composition can further comprise one or more phospholipids, for example, 1,2-dipalmitoyl-sn-glycero-3-phosphocholine (DPPC) or 1,2-distearoyl-sn-glycero-3-phosphocholine (DSPC) prior to spray drying in amounts up to about 25% (w/w), ranging from about 1% (w/w) to about 25%, or 2.5% to 20% (w/w), or 5% to 15% (w/w) for aiding in aerosolizing the formulation by decreasing the density of the particles. Yet in certain embodiments, in certain embodiments, in addition to imanitib or its salt, such as imatinib mesylate, and diketopiperazine, such as (E)-3,6-bis[4-(N-carbonyl-2-propenyl)amidobutyl]-2,5-diketopiperazine (FDKP), the dry powder composition may include a surfactant. In some embodiments, the dry powder composition may include only to imanitib or its salt, such as imatinib mesylate, diketopiperazine, such as (E)-3,6-bis[4-(N-carbonyl-2-propenyl)amidobutyl]-2,5-diketopiperazine (FDKP), and a surfactant.

In some embodiments, a dry powder inhaler may be, for example, a dry powder inhaler disclosed in WO2010/152477 or in U.S. Pat. No. 8,636,001, each of which is incorporated herein by reference in its entirety. Uses of dry powder inhalers for delivering compositions comprising diketopiperazine, such as (E)-3,6-bis[4-(N-carbonyl-2-propenyl)amidobutyl]-2,5-diketopiperazine (FDKP), are disclosed, for example, in WO2019/237028 and U.S. Pat. No. 8,508,732, each of which is incorporated herein by reference in its entirety.

The dry powder composition may have an average particle size less than 10 micrometers; or less than 9 microns or less than 8 microns or less than 7 microns or less than 6 microns or less than 5 microns or less than 4 microns or less than 3 micrometers in diameter. Particles size may be determined using a number of techniques, including laser diffraction technique. In some embodiments, the dry powder composition may have an average particle size from 0.5 microns to 10 microns or from 1 micron to 8 microns or from 1 micron to 5 microns or from 1 micron to 4 microns or from 1.5 microns to 4 microns or from 2 microns to 3 microns or any value or subrange within these ranges. In some embodiments, 50% of particles in the dry powder composition may have a size of less than 10 microns or less than 8 microns or less than 7 microns or less than 6 microns or less than 5 microns or less than 4 microns. In some embodiments, 90% of particles in the dry powder composition may have a size of less than 10 microns or less than 8 microns or less than 7 microns or less than 6 microns or less than 5 microns.

In some embodiments, 16% of particles in the dry powder composition when discharged from a dry powder inhaler, such as a dry powder inhaler disclosed in WO2010/152477 or in U.S. Pat. No. 8,636,001, may have size less than 4 microns or less than 3.5 microns or less than 3 microns or less than 2.5 microns or less or less than 2 microns. For example, 16% of particles in the dry powder composition when discharged from the dry powder inhaler may have a size from 0.5 microns to 4 microns or from 0.5 microns to 3.5 microns or from 0.5 microns to 3.0 microns or from 0.5 microns to 2.5 microns or from 0.5 microns to 2 microns. In some embodiments, 50% of particles in the dry powder composition when discharged from the dry powder inhaler may have size less than 20 microns or less than 18 microns or less than 15 microns or less than 12 microns or less or less than 10 microns or less than 8 microns or less than 7 microns or less than 6 microns or less than 5 microns. For example, 50% of particles in the dry powder composition when discharged from the dry powder inhaler may have a size from 0.5 microns to 20 microns or from 0.5 microns to 15 microns or from 0.5 microns to 10 microns or from 0.5 microns to 8 microns or from 0.5 microns to 7 microns or from 0.5 microns to 6 microns or from 0.5 microns to 5 microns. In some embodiments, 84% of particles in the dry powder composition when discharged from the dry powder inhaler may have size less than 60 microns or less than 55 microns or less than 50 microns or less than 45 microns or less or less than 40 microns or less than 35 microns or less than 30 microns or less than 25 microns or less than 22 microns or less than 21 microns or less than 20 microns. For example, 84% of particles in the dry powder composition when discharged from the dry powder inhaler may have a size from 0.5 microns to 60 microns or from 0.5 microns to 50 microns or from 0.5 microns to 45 microns or from 0.5 microns to 40 microns or from 0.5 microns to 35 microns or from 0.5 microns to 30 microns or from 0.5 microns to 25 microns or from 0.5 microns to 22 microns or from 0.5 microns to 21 microns or from 0.5 microns to 20 microns.

In some embodiments, the dry powder comprises an amorphous powder, a plurality of crystalline particles, or substantially homogenous crystalline composite particles. In some embodiments, the dry powder composition comprises amorphous, crystalline or crystalline composite particles made from a diketopiperazine, such as a compound having the formula:

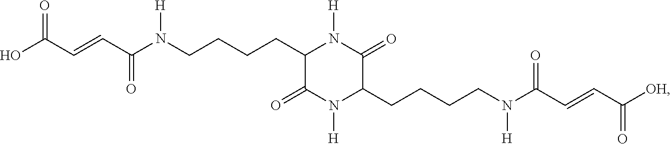 [SMILES: O=C(O)/C=C/C(=O)NCCCCC1NC(=O)C(CCCCNC(=O)/C=C/C(=O)O)NC1=O]

(E)-3,6-bis[4-(N-carbonyl-2-propenyl)amidobutyl]-2,5-diketopiperazine, or a pharmaceutically acceptable salt thereof, including, a disodium salt, a magnesium salt, a lithium and a potassium salt.

In some embodiments, the composition, such as a dry powder composition, can be administered to a patient in need in amounts from about 1 mg to about 800 mg or from about 1 mg to about 200 mg or from about 1 mg to about 100 mg or from about 0.15 mg to about 50 mg of the composition or imatinib or its pharmaceutically acceptable salt administered total weight per dose in one or more inhalations using an inhalation device, such as a dry powder inhaler. In certain embodiments, the total weight of the composition, such as a dry powder composition can range from about 1 mg to about 200 mg or from about 1 mg to about 100 mg or from about 5 mg to about 80 mg or from about 1 mg to 30 mg; 2 mg to 20 mg, or 3 mg to 10 mg the composition or imatinib or its pharmaceutically acceptable salt per single dose and/or per single administering event. In some embodiments, a daily dose of imatinib or its pharmaceutically acceptable salt administered using an inhalation device, such as a dry powder inhaler, may be from about 1 mg to about 800 mg or from about 1 mg to about 200 mg or from about 1 mg to about 100 mg or from about 1 mg to about 50 mg of the composition or imatinib or its pharmaceutically acceptable salt. The daily dose may be administered in one or multiple, e.g. two, three, four, five, etc. single administering events.

In some embodiments, the dry powder inhaler may deliver a dose of imatinib to lungs of a patient (“a lung dose”) from 0.1 mg to 200 mg or from 1 mg to 100 mg or from 5 mg to 80 mg or from 0.2 mg to 30 mg; 0.3 mg to 20 mg, or 0.5 mg to 10 mg. A dose of imatinib delivered to lungs of a patient via the dry powder inhaler may be effective to treat a pulmonary condition, such as pulmonary hypertension. For example, the effective dose may allow the subject with pulmonary hypertension to increase 6 minute walking distance by at least 5 m or at least 10 m or at least 20 m. In some embodiments, after the treatment, the subject may be able to walk at least 100 m during the 6 minutes walk test.

In some embodiments, the dry powder inhaler may deliver a dose of imatinib to lungs of a patient (“a lung dose”) from 0.1 mg/kg (of patient's weight (mass)) to 50 mg/kg or from 0.5 mg/kg to 25 mg/kg or from 1 mg/kg to 20 mg/kg or from 1 mg/kg to 10 mg/kg or from 2 mg/kg to 10 mg/kg or any value or subrange within these ranges.

In some embodiments, the dry powder inhaler may deliver a dose of imatinib to lungs of a patient to provide a lung concentration of imatinib of at least 20 ng/ml or at least 50 ng/ml or at least 100 ng/ml or at least 200 ng/ml or at least 300 ng/ml or at least 400 ng/ml or at least 500 ng/ml or at least 600 ng/ml or at least 700 ng/ml or at least 800 ng/ml or at least 1000 ng/ml or at least 1200 ng/ml or at least 1500 ng/ml or at least 1800 ng/ml or at least 2000 ng/ml or at least 2200 ng·ml or at least 2500 ng/ml or at least 2800 ng/ml or at least 3000 ng/ml or at least 3500 ng/ml or at least 4000 ng/ml or at least 4500 ng/ml.

In some embodiments, the dry powder inhaler may deliver a dose of imatinib to lungs of a patient to provide a lung concentration of imatinib of at least 20 ng/ml or at least 50 ng/ml or at least 100 ng/ml or at least 200 ng/ml or at least 300 ng/ml or at least 400 ng/ml or at least 500 ng/ml or at least 600 ng/ml one hour after the administering event.

In some embodiments, the dry powder inhaler may deliver a dose of imatinib to lungs of a patient to provide a lung concentration of imatinib of at least 20 ng/ml or at least 30 ng/ml or at least 40 ng/ml or at least 50 ng/ml or at least 60 ng/ml five hours after the administering event.

In some embodiments, the dry powder inhaler may deliver a dose of imatinib to lungs of a patient to provide a lung concentration of imatinib of at least 10 ng/ml or at least 15 ng/ml or at least 20 ng/ml or at least 21 ng/ml or at least 22 ng/ml or at least 23 ng/ml or at least 24 ng/ml or at least 25 ng/ml or at least 26 ng/ml or at least 27 ng/ml eight hours after the administering event.

In some embodiments, the dry powder composition can contain from about 1 wt % to about 60 wt % or 2 wt % to 55 wt % or 2.5 wt % to 50 wt % or from 5 wt % to 50 wt % or from 10 wt % to 40 wt % or a value or subrange within these ranges of imatinib or its salt, such as imatinib mesylate. In some embodiment, the dry powder composition comprises from about 5 wt % to about 50 wt %, or from about 5 wt % to about 30 wt %, or from about 10 wt % to about 20 wt % imatinib or its salt, such as imatinib mesylate, and diketopiperazine, such as (E)-3,6-bis[4-(N-carbonyl-2-propenyl)amidobutyl]-2,5-diketopiperazine particles. In some embodiments, the dry powder composition comprises crystalline composite carrier particles which are made by spray drying a suspension of the particles and imatinib or its salt, such as imatinib mesylate, In some embodiments, such composition may comprise up to about 800 μg or about 5 mg or about 10 mg or about 20 mg or about 40 mg or about 80 mg of imatinib per dose, which can be provided in a capsule or cartridge for a dry powder inhaler. In some embodiments, a dose of the composition or imatinib or its salt provided in a container, such as a capsule or a cartridge, for a dry powder inhaler may be from 1 mg to 200 mg or from 1 mg to 150 mg or from 1 mg to 100 mg or from 5 mg to 80 mg.

The metered dose inhaler can be a soft mist inhaler (SMI), in which the aerosol cloud containing a respiratory drug can be generated by passing a solution containing the respiratory drug through a nozzle or series of nozzles. The aerosol generation can be achieved in SMI, for example, by mechanical, electromechanical or thermomechanical process. Examples of soft mist inhalers include the Respimat® Inhaler (Boeringer Ingelheim GmbH), the AERx® Inhaler (Aradigm Corp.), the Mystic™ Inhaler (Ventaira Pharmaceuticals, Inc.) and the Aira™ Inhaler (Chrysalis Technologies Incorporated). For a review of soft mist inhaler technology, see e.g. M. Hindle, The Drug Delivery Companies Report, Autumn/Winter 2004, pp. 31-34. The aerosol for SMI can be generated from a solution of the respiratory drug further containing pharmaceutically acceptable excipients. In the present case, the respiratory drug is imatinib, its derivative or a pharmaceutically acceptable salt thereof, which can be formulated in SMI as a solution. The solution can be, for example, a solution of imatinib in water, ethanol or a mixture thereof. Preferably, the diameter of the imatinib-containing aerosol particles is less than about 10 microns, or less than about 5 microns, or less than about 4 microns.

Imatinib, a pharmaceutically acceptable salt, or derivative thereof concentration in an aerosolable composition, such as a dry powder, used in a metered dose inhaler or dry powder inhaler can range from about 500 μg/g to about 2500 μg/g, or from about 800 μg/g to about 2200 μg/g, or from about 1000 μg/g to about 2000 μg/g (concentrations in μg of imatinib/g of dry powder). Imatinib, a pharmaceutically acceptable salt, or derivative thereof concentration in an aerosolable composition, such as a solution, used in a metered dose inhaler can range from about 500 μg/ml to about 2500 μg/ml, or from about 800 μg/ml to about 2200 μg/ml, or from about 1000 μg/ml to about 2000 μg/ml.

The dose of imatinib, a pharmaceutically acceptable salt, or derivative thereof that can be administered using an inhalation device, such as a dry powder inhaler, in a single event (a single pump or discharge of the inhaler) can be up to about 1 mg to about 200 mg or about 1 mg to about 100 mg or about 1 mg to about 50 mg total (of dry powder or of imatinib). In certain embodiments, the total weight of the dry powder composition administered by a single event can range from about 1 mg to about 200 mg or from about 1 mg to about 100 mg or from about 5 mg to about 80 mg or about 1 mg to 30 mg; 2 mg to 20 mg, or 3 mg to 10 mg per dose (of dry powder or of imatinib).

The pharmaceutically effective amount of imatinib, a pharmaceutically acceptable salt, or derivative thereof in the methods can be, for example, about 0.1 mg to about 1 mg, about 1 mg to about 5 mg, about 5 mg to about 10 mg, about 10 mg to about 20 mg, about 20 mg to about 50 mg, about 50 mg to about 100 mg, or about greater than 100 mg. Effective amounts of imatinib can be provided in one or more capsules or cartridges for use with a corresponding dry powder inhaler.

Of course, the dosage may be changed according to the subject's age, weight, species, susceptibility, symptom, or the efficacy of the treatment.

Administering of imatinib, a pharmaceutically acceptable salt, or derivative thereof in a single event can be carried out in a limited number of breaths by a patient. For example, imatinib can be administered in 20 breaths (inhalations) or less, or 19 breaths or less, or 18 breaths or less, or 17 breaths or less, or 16 breaths or less, or 15 breaths or less or 14 breathes or less, or 13 breaths or less, or 12 breaths or less, or 11 breaths or less, or 10 breaths or less, or 9 breaths or less, or 8 breaths or less, or 7 breaths or less, or 6 breaths or less, or than 5 breaths or less, or 4 breaths. For example, imatinib may be administered in 3, 2 or 1 breaths. The total time of a single administering event can be less than 5 minutes, or less than 4 minutes or less than 3 minutes or less than 2 minutes or less than 1 minute, or less than 45 seconds or less than 30 seconds or less than 20 seconds. Imatinib, a pharmaceutically acceptable salt, or derivative thereof can be administered a single time (single administering event) per day or several times (single administering events), such as 2, 3 or 4 times, per day.

In yet another embodiment, imatinib, a pharmaceutically acceptable salt or derivative thereof is administered once every about fifth of a day, about fourth of a day, about third of a day, about half a day, about 1, about 2, about 3, about 4, about 5, about 6, about 7, about 8, about 9, about 10, about 11, about 12, about 13, or about 14 days. In another embodiment, imatinib, a pharmaceutically acceptable salt or derivative thereof is given once per day, every other day, once per week, twice per week, three times per week, four times per week, five times per week, six times per week, every other week, or every few days.

In some embodiments, the method may result in reduction or elimination of one or more symptoms of pulmonary hypertension. The symptom may be selected from dyspnea, fatigue, dizziness, chest pain, edema, cyanosis, and heart palpitation. The reduction may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by one or more medically recognized techniques.

In some embodiments, administering does not comprise a systemic side effect on the subject. In some embodiments, a systemic side effect is reduced as compared to a non-dry powder inhalation administration of the same dose or amount to the subject. The systemic side effect may be selected from one or more of subdural hematoma, edema, upset stomach, musculoskeletal pain, muscle cramp, dizziness, blurred vision, anorexia, vomiting, diarrhea, hemoglobin decrease, rash, and drowsiness.

In some embodiments, the reduction in systemic side effect as compared to a non-dry powder inhalation administration is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by one or more medically recognized techniques. The non-dry powder administration may be selected from oral, nasal, sublingual, buccal, intravenous, intramuscular, transdermal, liquid or gas aerosol inhalation, rectal, or vaginal.

The medically recognized technique may be selected from the Borg scale, numerical rating scale, visual analogue scale, Fatigue Severity Scale, Dizziness Assessment Rating Scale (DARS), SVEAT Chest Pain Scoring System, EKG, Holter monitor, Epworth Sleepiness Scale (ESS), computed tomography (CT scan), magnetic resonance imaging (MRI scan), adult diarrhea state score (ADSS), Chronic Subdural Hematoma grading system as described in Stanišić et al., Neurosurgery. 2017 November; 81(5):752-760 or edema assessment through methods 1-8 of (1) clinical assessment of pit depth and recovery at three locations, (2) patient questionnaire, (3) ankle circumference, (4) figure-of-eight (ankle circumference using eight ankle/foot landmarks), (5) edema tester (plastic card with holes of varying size pressed to the ankle with a blood pressure cuff), (6) modified edema tester (edema tester with bumps), (7) indirect leg volume (by series of ankle/leg circumferences), and (8) foot/ankle volumetry by water displacement.

In some embodiments, the method of treatment of pulmonary hypertension can further comprise administering at least one supplementary active agent selected from the group consisting of prostacycicins, such as flolan, iloprost, beraprost or treprostinil, sildenafil, tadalafil, calcium channel blockers (diltiazem, amlodipine, nifedipine), bosentan, sitaxsentan, ambrisentan, and pharmaceutically acceptable salts thereof. In some embodiments, the supplementary active agents can be included in the imatinib composition and thus can be administered simultaneously with imatinib using an inhalation device, such as a dry powder inhaler. In some embodiments, the supplementary agents can be administered separately from imatinib. In some embodiments, the application of intravenous prostacyclin (flolan), intravenous, subcutaneous, oral or inhaled treprostinil, intravenous iloprost or intravenous or subcutaneous imatinib can be administered in addition to imatinib administered via inhalation.

Compositions and Methods of Making the Same

In another aspect a dry powder inhalable composition is provided, the composition comprising imatinib, a pharmaceutically acceptable salt, or a derivative thereof, and optionally one or more excipients.

Regarding the composition in its solid dry form, the excipient also forms the solid matrix in which the imatinib, a salt, or derivative thereof is dispersed. In a preferred embodiment, the main excipient is (E)-3,6-bis[4-(N-carbonyl-2-propenyl)amidobutyl]-2,5-diketopiperazine, fumaryl diketopiperazine (FDKP), or a salt thereof. The excipient may be processed to obtain crystals of an appropriate size to form crystalline powders, crystalline composite powders, or dissolved to obtain amorphous powders.

The composition may include excipients such as lactose, corn starch, or the like, glidants such as magnesium stearate, etc., emulsifying agents, suspending agents, stabilizers, and isotonic agents, etc. If desired, a sweetening agent and/or a flavoring agent may be added. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like. Other examples of soluble excipients that may be used in the composition are alitame, acesulfame potassium, aspartame, saccharin, sodium saccharin, sodium cyclamate, sucralose, threalose, xylitol, citric acid, tartaric acid, cyclodextrins, dextrins, hydroxyethylcellulose, gelatine, malic acid, maltitol, maltodextrin, maltose, polydextrose, tartaric acid, sodium or potassium bicarbonate, sodium or potassium chloride, sodium or potassium citrate, phospholipids, lactose, sucrose, glucose, fructose, mannitol, sorbitol, natural aminoacids, alanine, glycine, serine, cysteine, phenylalanine, tyrosine, tryptophan, histidine, methionine, threonine, valine, isoleucine, leucine, arginine, lysine, aspartic acid, glutamic acid, asparagine, glutamine, proline, their salts, and their possible simple chemical modifications such as in N-acetylcysteine, and carbocysteine.

The preferred soluble excipients are alkaline metals salts such as sodium chloride or potassium chloride, and sugars, such as lactose. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

In some embodiments, the excipient comprises a surfactant. The surfactant of the composition can be chosen among different classes of surfactants of pharmaceutical use.

Surfactants suitable to be used are all those substances characterized by medium or low molecular weight that contain a hydrophobic moiety, generally readily soluble in an organic solvent but weakly soluble or insoluble in water, and a hydrophilic (or polar) moiety, weakly soluble or insoluble in an organic solvent but readily soluble in water. Surfactants are classified according to their polar moiety. Therefore surfactant with a negatively charged polar moiety are called anionic surfactants, while cationic surfactants have a positively charged polar moiety. Uncharged surfactant are generally called non-ionic, while surfactant charged both positively and negatively are called zwitterionic. Examples of anionic surfactants are salts of fatty acids (better known as soaps), sulfates, sulfate ethers and phosphate esters. Cationic surfactants are frequently based on polar groups containing amino groups. Most common non-ionic surfactants are based on polar groups containing oligo-(ethylene-oxide) groups. Zwitterionic surfactants are generally characterized by a polar group formed by a quaternary amine and a sulfuric or carboxylic group.

Specific examples of this application are the following surfactants: benzalkonium chloride, cetrimide, docusate sodium, glyceryl monolaurate, sorbitan esters, sodium lauryl sulfate, polysorbates, phospholipids, biliary salts.

Non-ionic surfactants, such as polysorbates and polyethylene and polyoxypropylene block copolymers, known as “Poloxamers,” may be used. Polysorbates are described in the CTFA International Cosmetic Ingredient Dictionary as mixtures of sorbitol and sorbitol anhydride fatty acid esters condensed with ethylene oxide. Particularly preferred are non-ionic surfactants of the series known as “Tween,” in particular the surfactant known as “Tween 80,” a polyoxyethylensorbitan. Additional exemplary excipients include surfactants such as other polysorbates, e.g., “Tween 20” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

The presence of a surfactant, and preferably of Tween 80, may be necessary to reduce electrostatic charges found in compositions without it, the flow of the powder and the maintenance of the solid state in a homogeneous way without initial crystallization. Phospholipids may be included in the above mentioned definition of surfactants or excipients.

The inhalatory formulation can include a hydrophobic substance in order to reduce sensitivity to humidity. Such hydrophobic substance is preferably leucine, which makes the particle disaggregation easier.

In case of production of a solid product in powder form, this can occur using different techniques, well consolidated in the pharmaceutical industry. The preparation of fine particles through spray-drying may one exemplary embodiment. In case of industrial production, this technique is undoubtedly preferred to freeze-drying, which at the moment is the most expensive drying process, both for the apparatus used, and for the yield and production times.

The pharmaceutical composition can include other components, such as pH buffers and preservatives. Buffers include, but are not limited to, citric acid, sodium chloride, potassium chloride, sodium sulfate, potassium nitrate, sodium phosphate monobasic, sodium phosphate dibasic, and combinations thereof.

Further, a composition disclosed herein may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Non-limiting examples of suitable bases include bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The excipients may include an antioxidant, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The term “dry powder” refers to a powder, granulate, tablet form composition, or any other solid form with a humidity content that assures to the composition chemical stability in time. More precisely, the term “dry” refers to a solid composition with water content lower than 10% w/w, normally less than 5% and preferably less than 3%.

The amount of any excipient in the dry powder composition can change within a wide range. The amount of any individual excipient in the composition will vary depending on the role of the excipient, the dosage requirements of the active agent components, and particular needs of the composition. Generally, however, the excipient will be present in the composition in an amount of about 1% to about 99% by weight, preferably from about 5% to about 98% by weight, more preferably from about 15% to about 95% by weight of the excipient. In general, the amount of excipient present in a composition of the disclosure is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

The present disclosure also provides a kit that includes a dry powder inhaler and pre-filled unit-dose cartridges containing a pharmaceutical composition comprising imatinib or its derivative, or a pharmaceutically acceptable salt thereof. Such a kit can further include instructions on how to use the inhaler for inhaling imatinib. The kit can be used by a subject, such as human being, affected with a disease or condition that can be treated by imatinib, such as asthma, pulmonary hypertension, peripheral vascular disease, or pulmonary fibrosis.

In some cases, the kit is a kit for treating pulmonary hypertension that includes (i) an inhalation device, such as a dry powder inhaler, and pre-filled unit-dose cartridges containing a pharmaceutical composition comprising imatinib or its derivative, or a pharmaceutically acceptable salt thereof; and (ii) instructions for use of the inhalation device, such as a dry powder inhaler, containing imatinib in treating pulmonary hypertension. In certain embodiments, the kit can comprise blisters containing multiple pre-filled unit-dose cartridges.

### Definitions

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “comprising” or “comprises” is intended to mean that the compositions and methods include the recited elements, but do not exclude others. A composition or method “consisting essentially” of the elements as defined herein would not exclude other materials or steps that do not materially affect the basic and novel characteristic(s) of the claimed technology. “Consisting of” shall mean excluding more than trace elements of other ingredients and substantial method steps. Embodiments defined by each of these transition terms are within the scope of this technology. When an embodiment is defined by one of these terms (e.g., “comprising”) it should be understood that this disclosure also includes alternative embodiments, such as “consisting essentially of” and “consisting of” for said embodiment.

“Pulmonary hypertension” refers to all forms of pulmonary hypertension, WHO Groups 1-5. Pulmonary arterial hypertension, also referred to as PAH, refers to WHO Group 1 pulmonary hypertension. PAH includes idiopathic, heritable, drug- or toxin-induced, and persistent pulmonary hypertension of the newborn (PPHN).

“Subdural hematoma” or SDH, as used herein, refers to a type of bleeding in which a collection of blood gathers between the inner layer of the dura mater and the arachnoid mater of the meninges surrounding the brain.

“Edema” as used herein, refers to swelling, for example, of a body part of the subject.

“Non-dry powder inhalation administration” as used herein refers to any route of administration that does not comprise inhalation of a dry powder formulation. Examples include oral, nasal, sublingual, buccal, intravenous, intramuscular, transdermal, liquid or gas aerosol inhalation, rectal, or vaginal administration.

“Subject” refers to an animal, such as a mammal (including a human), that has been or will be the object of treatment, observation or experiment. “Subject” and “patient” may be used interchangeably, unless otherwise indicated. The methods described herein may be useful in human therapy and/or veterinary applications. In some embodiments, the subject is a mammal. In some embodiments, the subject is a human.

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound disclosed herein for the purpose of (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

Unless defined otherwise, all technical and scientific terms used herein have the same meaning as commonly understood by one of ordinary skill in the art to which this present technology belongs. Although any methods and materials similar or equivalent to those described herein can also be used in the practice or testing of the present technology, representative illustrative methods and materials are described herein.

As used herein, the phrase “instructions for use” shall mean any FDA-mandated labeling, instructions, or package inserts that relate to the administration of imatinib or its derivatives, or pharmaceutically acceptable salts thereof, for treatment of pulmonary hypertension by inhalation. For example, instructions for use may include, but are not limited to, indications for pulmonary hypertension, identification of specific symptoms associated with pulmonary hypertension, that can be ameliorated by imatinib, recommended dosage amounts for subjects suffering from pulmonary hypertension and instructions on use of an inhalation device, such as a dry powder inhaler, and cartridges, or on coordination of individual's breathing and actuation with use of the inhalation device.

As used herein “derivative” may refer a compound described in U.S. Pat. No. 5,521,184, the disclosure of which is hereby incorporated by reference, or a compound corresponding to imatinib wherein: one or more aromatic N is replaced with CR¹; one or more aromatic CH is replaced with N; one or more CH is replaced with CR¹; one or more NH is replaced with O, S, or NR¹; one or more tertiary non-aromatic N is replaced with CR¹; and/or one or more aryl group of imatinib is replaced with a different aryl or heteroaryl group; wherein each R¹ is independently hydroxyl, optionally substituted amino, halo, optionally substituted C₁-C₆ alkyl, optionally substituted C₂-C₆ alkenyl, optionally substituted C₂-C₆ alkynyl, optionally substituted C₃-C₇ cycloalkyl, optionally substituted C₃-C₇ hetereocyclyl, optionally substituted C₁-C₆ alkoxy, optionally substituted aryl, acyloxy, acylamino, acyl, or optionally substituted heteroaryl.

“Substituted” may refer to substitution with any of the groups defined below.

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused, bridged and spiro ring systems. In fused ring systems, one or more of the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Halo” or “halogen” refers to fluoro, chloro, bromo, and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N oxide (N→O), sulfinyl, or sulfonyl moieties. Certain non-limiting examples include pyridinyl, pyrrolyl, indolyl, thiophenyl, oxazolyl, thizolyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

Examples of heterocycle and heteroaryls include, but are not limited to, azetidine, pyrrole, furan, thiophene, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4 tetrahydroisoquinoline, 4,5,6,7 tetrahydrobenzo[b]thiophene, thiazole, thiazolidine, thiophene, benzo[b]thiophene, morpholinyl, thiomorpholinyl (also referred to as thiamorpholinyl), 1,1 dioxothiomorpholinyl, piperidinyl, pyrrolidine, and tetrahydrofuranyl.

“Cycloalkyl” refers to cyclic alkyl groups of from 3 to 10 carbon atoms having single or multiple cyclic rings including fused, bridged, and spiro-ring systems. The fused ring can be an aryl ring provided that the non-aryl part is joined to the rest of the molecule. Examples of suitable cycloalkyl groups include, for instance, adamantyl, cyclopropyl, cyclobutyl, cyclopentyl, and cyclooctyl.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2 benzoxazolinone, 2H 1,4 benzoxazin 3(4H) one 7 yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Optionally substituted” refers to a group selected from that group and a substituted form of that group. Substituents may include any of the groups defined below. In one embodiment, substituents are selected from C₁-C₁₀ or C₁-C₆ alkyl, substituted C₁-C₁₀ or C₁-C₆ alkyl, C₂-C₆ alkenyl, C₂-C₆ alkynyl, C₆-C₁₀ aryl, C₃-C₈ cycloalkyl, C₂-C₁₀ heterocyclyl, C₁-C₁₀ heteroaryl, substituted C₂-C₆ alkenyl, substituted C₂-C₆ alkynyl, substituted C₆-C₁₀ aryl, substituted C₃-C₈ cycloalkyl, substituted C₂-C₁₀ heterocyclyl, substituted C₁-C₁₀ heteroaryl, halo, nitro, cyano, —CO₂H or a C₁-C₆ alkyl ester thereof.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂), and neopentyl ((CH₃)₃CCH₂—).

“Alkenyl” refers to monovalent straight or branched hydrocarbyl groups having from 2 to 10 carbon atoms and preferably 2 to 6 carbon atoms or preferably 2 to 4 carbon atoms and having at least 1 and preferably from 1 to 2 sites of vinyl (>C═C<) unsaturation. Such groups are exemplified, for example, by vinyl, allyl, and but 3-en-1-yl. Included within this term are the cis and trans isomers or mixtures of these isomers.

“Alkynyl” refers to straight or branched monovalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Alkoxy” refers to the group O alkyl wherein alkyl is defined herein. Alkoxy includes, by way of example, methoxy, ethoxy, n propoxy, isopropoxy, n butoxy, t butoxy, sec butoxy, and n pentoxy.

“Substituted alkoxy” refers to the group O (substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl —C(O)O, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, SO₂ alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology.

The present invention can be illustrated in more detail by the following examples, however, it should be understood that the present invention is not limited thereto.

As will be apparent to those of skill in the art upon reading this disclosure, each of the individual embodiments described and illustrated herein has discrete components and features which may be readily separated from or combined with the features of any of the other several embodiments without departing from the scope or spirit of the present technology. Any recited method can be carried out in the order of events recited or in any other order which is logically possible.

### EXAMPLES

### Example 1: Preparation of Imatinib Mesylate Powders for Inhalation

Imatinib inhalation powders were prepared as Technosphere® (T) powders or crystalline carrier (XC) powders. T particles were formed by the crystallization of FDKP in the presence of surfactant Tween 20 and subsequent self-assembly to form a suspension of particles approximately 2-2.5 μm in diameter. XC particles were formed by spray drying a suspension of FDKP crystals formed under conditions where they do not self-assemble in suspension into particles. A 25% solution of imatinib mesylate was prepared in deionized water and added to either a T suspension of preformed particles, or to an XC suspension comprising crystallites to prepare powders containing 2.5 wt % to 50 wt % imatinib on a dry basis (Table 1A). The T and XC suspensions containing imatinib mesylate were dried either by lyophilization or spray drying. T suspensions containing 10% and 20% imatinib mesylate were lyophilized after being pelletized into liquid nitrogen. The lyophilizer shelf temperature was increased from −45° C. to 25° C. at 0.2° C./min and maintained at 25° C. under vacuum until the powder was completely dried. All other imatinib mesylate T and XC powders were prepared by spray drying with a Buchi B-290 spray dryer operating at an inlet temperature of 180° C., an aspirator pump speed of 90%, feed pump speed of 25% and a nitrogen flow rotameter reading of 60 mm.

TABLE 1A
Preparation and Composition of Imatinib Mesylate Powders

| | | Suspension | | HPLC |
|---|---|---|---|---|
| Powder Description | Drying | Solids | % | Assay |
| (API %) | Technique | (%) | Yield | (wt %) |
| 50% imatinib T powders | Spray Dried | 8.11 | 77.2 | 48.3 |
| 50% Unwashed XC powder | Spray Dried | 1.49 | 49.6 | 47.45 |
| 50% imatinib XC powder | Spray Dried | 1.58 | 82 | 43.69 |
| 30% imatinib XC powder | Spray Dried | 1.64 | 67.6 | 30.08 |
| 40% imatinib XC powder | Spray Dried | 1.64 | 72.8 | 40.81 |
| 20% imatinib XC powder | Spray Dried | 1.64 | 51.2 | 20.34 |
| 10% imatinib XC powder | Spray Dried | 1.31 | 62 | 8.65 |
| 10% imatinib XC powder | Spray Dried | 1.31 | 63.2 | 8.74 |
| 5% imatinib XC powder | Spray Dried | 1.31 | 60.08 | 4.38 |
| 2.5% imatinib XC powder | Spray Dried | 1.31 | 61.6 | 2.17 |
| 20% imatinib XC powder | Spray Dried | 1.31 | 47.2 | 18.11 |
| 10% imatinib XC powder | Spray Dried | 1.31 | 69.2 | 9.6 |
| 20% imatinib T powder | lyophilized | 8.11 | 87.6 | 16.04 |
| 10% imatinib T powder | Lyophilized | 8.11 | 93.2 | 10.24 |

### Example 2: Imatinib Powder Particle Size and Geometry

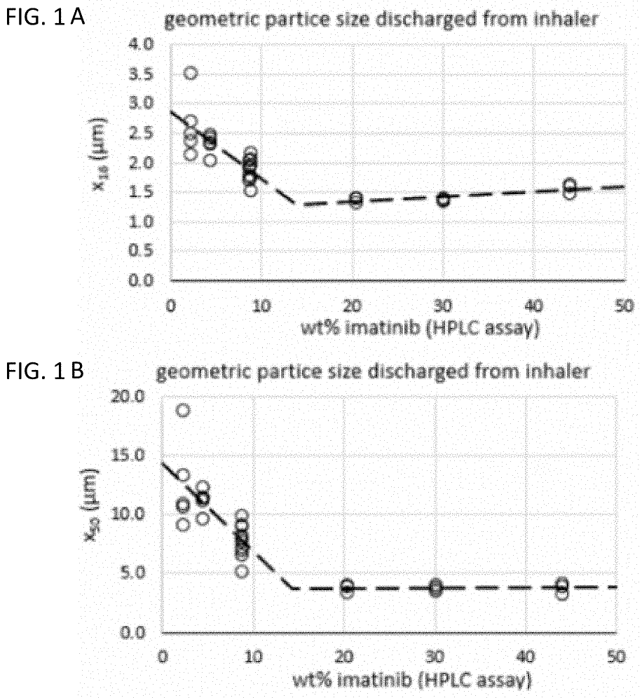

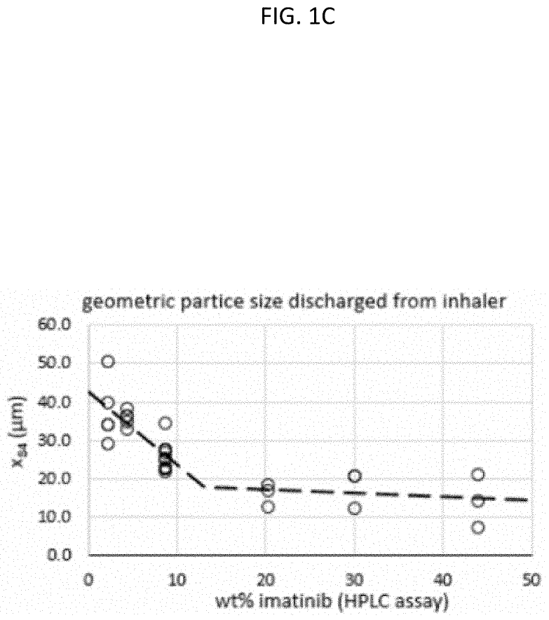

Powders were evaluated for geometric particle size distribution using a Sympatec laser diffraction instrument fitted with either a RODOS™ powder dispersing system or an inhaler adapter. Bulk powders were dispersed using the RODOS™ powder dispersing system at either 0.5 bar or 3.0 bar. When using the inhaler adapter, 10 mg samples of powders were discharged from Dreamboat Gen2C dry powder inhalers (MannKind Corp.—See U.S. Pat. No. 8,508,732, incorporated herein by reference) at 4 kPa. Samples evaluated with the anatomically correct airway were also discharged from a Gen2C inhaler filled with 10 mg of powder at 4 kPa. These data are shown in Table 2. FIGS. 1A-1C plot the Sympatec inhaler data for the 16^{th}, 50^{th}, and 84^{th} percentiles (x₁₆, x₅₀, and x₈₄) of the particle size distribution for imatinib XC powders from Table 2.

TABLE 2
Geometric Particle Size Data for Imatinib Mesylate Powders

| | Sympatec Data | | | | | | | |
|---|---|---|---|---|---|---|---|---|
| | RODOS Data | | | | Inhaler Data | | | |
| | 0.5 bar | | 3.0 bar | | Avg. | | | |
| Powder Description | x(50) | x(90) | x(50) | x(90) | CE | x(16) | x(50) | x(84) |
| (API %) | (μm) | (μm) | (μm) | (μm) | (%) | (μm) | (μm) | (μm) |
| 50% imatinib T powders | 3.47 | 7.25 | 2.79 | 6.78 | 27.40% | 2.54 | 5.00 | 9.26 |
| 50% Unwashed XC powder | 1.90 | 4.05 | 1.47 | 3.39 | 49.00% | 1.54 | 3.36 | 36.04 |
| 50% imatinib XC powder | 2.24 | 5.01 | 1.77 | 4.18 | 59.00% | 1.57 | 3.79 | 14.13 |
| 30% imatinib XC powder | 1.92 | 4.34 | 1.61 | 3.62 | 71.16% | 1.38 | 3.75 | 17.94 |
| 40% imatinib XC powder | 1.87 | 4.32 | 1.52 | 3.46 | 47.80% | 2.00 | 7.13 | 58.75 |
| 20% imatinib XC powder | 2.02 | 4.56 | 1.61 | 3.50 | 74.28% | 1.38 | 3.77 | 16.07 |
| 10% imatinib XC powder | 1.95 | 4.62 | 1.54 | 3.42 | 91.26% | 1.87 | 7.66 | 24.30 |
| 10% imatinib XC powder | 1.92 | 4.58 | 1.56 | 3.37 | 98.52% | 1.89 | 7.94 | 27.21 |
| 5% imatinib XC powder | 2.00 | 5.23 | 1.60 | 3.60 | 97.68% | 2.32 | 11.19 | 35.76 |
| 2.5% imatinib XC powder | 2.25 | 5.90 | 1.64 | 3.54 | 89.58% | 2.65 | 12.55 | 37.56 |
| 20% imatinib XC powder | 2.15 | 4.43 | 1.83 | 3.77 | 96.70% | 1.60 | 4.48 | 17.91 |
| 10% imatinib XC powder | 2.17 | 4.67 | 1.79 | 3.79 | 97.54% | 1.61 | 5.14 | 20.29 |
| 20% imatinib T powder | 2.33 | 6.37 | 1.53 | 3.15 | 87.54% | 2.46 | 9.19 | 25.78 |
| 10% imatinib T powder | 1.98 | 4.96 | 1.4 | 2.7 | 97.56% | 1.67 | 5.13 | 17.14 |

### Example 3: Imatinib Powder Aerodynamic Performance Testing

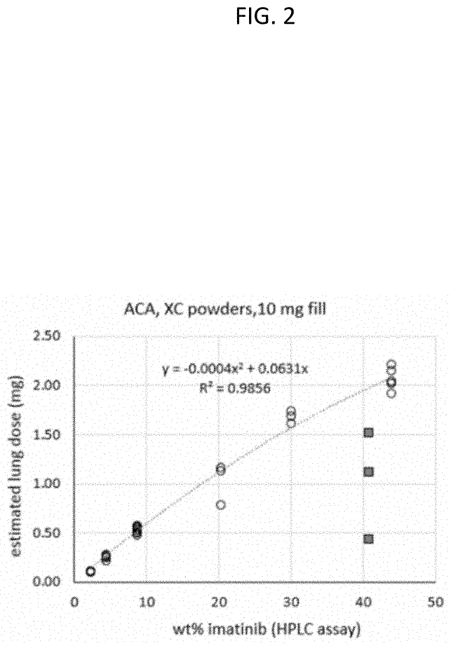

Aerodynamic performance was evaluated using MannKind's Anatomically Correct Airway (ACA, U.S. Pat. No. 9,706,944, incorporated by reference herein) which simulates inhalation efforts performed by a subject. The powder (10 mg) was filled into cartridges and the filled cartridge was weighed. The cartridges were inserted into the Dreamboat Gen2C inhaler and positioned into the mouth opening of a model of the upper airway of a male individual in his 20s. The powder was discharged into the airway with a 4 kPa pressure drop and a filter at the bottom of the airway collected any powder that passed through the oropharynx on its way to the lungs. The discharged cartridge was reweighed to determine the percentage of powder that was discharged (% CE, cartridge emptying). The filter was weighed to determine the amount of powder reaching the filter (MtF, mass-to-filter) and the result was normalized to the amount of powder filled in the cartridges (MtF/F, mass-to filter over fill). Results are presented in Table 3 and the estimated lung dose as a function of imatinib content is presented in FIG. 2. Estimated lung dose is the product of cartridge content, MtF/F, and imatinib content. For example, powder 963-119 (30.08 wt % imatinib, 55.90% MtF/F) would provide an estimated lung dose of (10 mg)(0.3008)(0.5590)=1.68 mg imatinib.

TABLE 3
Anatomically Correct Airway Results

| | ACA Data | |
|---|---|---|
| | Avg. | |
| | cartridge | |
| Powder Description | emptying | Avg. |
| (API %) | (CE) | MtF/F |
| 50% imatinib T | 73.40% | 28.90% |
| powders | | |
| 50% Unwashed XC | 55.10% | 28.00% |
| powder | | |
| 50% imatinib XC | 87.70% | 47.20% |
| powder | | |
| 30% imatinib XC | 94.70% | 55.90% |
| powder | | |
| 40% imatinib XC | 58.70% | 25.30% |
| powder | | |
| 20% imatinib XC | 89.50% | 50.50% |
| powder | | |
| 10% imatinib XC | 98.29% | 59.80% |
| powder | | |
| 10% imatinib XC | 96.86% | 62.90% |
| powder | | |
| 5% imatinib XC | 99.07% | 58.50% |
| powder | | |
| 2.5% imatinib XC | 98.01% | 51.10% |
| powder | | |
| 20% imatinib XC | 98.00% | 56.10% |
| powder | | |
| 10% imatinib XC | 96.88% | 63.90% |
| powder | | |
| 20% imatinib T powder | 98.39% | 48.70% |
| 10% imatinib T powder | 98.48% | 58.60% |

### Example 4

### Determination of the In Vivo Pharmacokinetics of Insufflated ImaT Powder in Rat

### SUMMARY

The pharmacokinetics of a dry powder formulation of Imatinib (ImaT) was evaluated in this rat study. After dosing, blood samples and lungs were collected at specific time points over a period of 24 hours. The target dose was 1 mg of ImaT per rat, delivered as an inhaled dry powder.

Target dose of test compound was based on information available at the time of the design of this study. The selected dose reflects one expected to exceed the therapeutic dose in humans.

### METHODS AND EXPERIMENTAL DESIGN

### 1. Test System

### 1.1 Species

Male Sprague Dawley rats (Charles River Laboratories) weighing between 225 and 275 grams at the time of their enrolment were used in the study.

### 1.2 Identification and Randomization of the Test System

1. The animals arrived at IPST at least 3 days prior to the planned experiment.

2. The animals were identified upon arrival as per CCAC guidelines.

3. All animal care and vivarium maintenance were recorded, with documents kept at the test facility.

4. The animals were randomly assigned to one of the groups before the experiment by the study director, who kept records of each animal's ID number.

### 1.3 Justification of the Test System

Sprague-Dawley rats were selected because they are recommended by various regulatory authorities and have been used frequently in inhaled dry powder studies.

### 2. Test Article and Reference Compound

### 2.1 Test Article

### Code Name: ImaT Inhalation Powder (20% Imatinib)

### Supplier: MannKind Corporation

Lot number: 963-123

### Retest Date: May 2021

Storage conditions: −20° C.
At the end of the study, all remaining test article was stored at −20±3° C.

### Dose Justification

Target dose of the test compound was based on information available at the time of the design of this study. The selected dose reflects one thought to exceed the therapeutic dose in human. ImaT was delivered as a target dose of 1 mg to rats weighing approximately 250 grams, leading to an average dose of 4 mg/kg.

### 3. Experimental Procedures

Male Sprague Dawley rats weighing between 225 and 275 grams arrived at the facility the week before the beginning of the experiment. The animals were pair-housed during the acclimation period.

After the acclimation period, each animal was randomly assigned to a dose treatment group (see Table 4).

TABLE 4
Experimental Design

| | | | Target | | | | Sacrifice |
|---|---|---|---|---|---|---|---|
| | | Target fill | amount of | | | | and |
| | | mass for | inhalation | | | PK | lung |
| | Test | Guppy | powder per | | | Bleeding | harvest |
| Group | article | (mg) | rat (mg) | Route | n | Time | time |
| 1 | ImaT | 1.2 | 1.0 | DPI | 4 | 5 min | 5 min |
| 2 | ImaT | 1.2 | 1.0 | DPI | 4 | 15 min | 1 h |
| | | | | | | 1 h | |
| 3 | ImaT | 1.2 | 1.0 | DPI | 4 | 30 min | 4 h |
| | | | | | | 4 h | |
| 4 | ImaT | 1.2 | 1.0 | DPI | 4 | 2 h | 8 h |
| | | | | | | 8 h | |
| 5 | ImaT | 1.2 | 1.0 | DPI | 4 | predose | 24 h |
| | | | | | | 24 h | |

The rats were anesthetized with a mixture of 1.5 to 3% isoflurane USP (Abbott Laboratories, Montreal Canada) in 100% oxygen, and placed on a homoeothermic heating pad to maintain body temperature at 37±1° C.

For all groups, the test article was insufflated using an automated insufflation device. The insufflator tip was inserted just above the bifurcation of the trachea and the discharge of powder was timed to the inhalation cycle of the animal. After dosing, the animals were allowed to recover from anesthesia under surveillance before being returned to their respective cages.

Blood samples of 0.5 mL were collected from the jugular vein at specified time points (see Table 4).

Blood samples were centrifuged (3000 r.p.m. for 10 min., 2-8° C.) and plasma was transferred into an Eppendorf tube labeled with the Study Number, animal I.D., dose group and time point. The plasma samples were stored frozen (−80° C.) until shipment to for plasma Imatinib content quantification.

Lungs were also harvested at specified time points (see Table 4). To do so, the animals were anesthetized with a mixture of 1.5 to 3% isoflurane USP (Abbott Laboratories, Montreal Canada) in 100% oxygen and were euthanized by exsanguination. Lungs werethen harvested. The left lobe was sectioned into three parts; upper, middle and bottom. Each section was separated into two equal pieces. Each piece was weighed and individually placed in a properly labeled tube. Lungs samples were snap-frozen and stored at −80° C. Lung samples were then homogenized in PBS, 0.1% Triton X-100 (200 mg of tissue/mL). The homogenate samples were stored at −80° C.

Plasma samples and lung homogenates were shipped on dry ice for testing according to the Sponsor's instructions.

### Control of Bias in the System

Any reported mishandling of the samples or animals led to final analysis exclusion.

### Data Calculations

Results were analyzed with Microsoft Excel 2010 using PK Solver Add-in as per Zhang et al. (2010), as well as Certara Phoenix WinNonLin 7.0. For each group, data in this report is expressed as mean±S.E.M.

### Results and Discussion

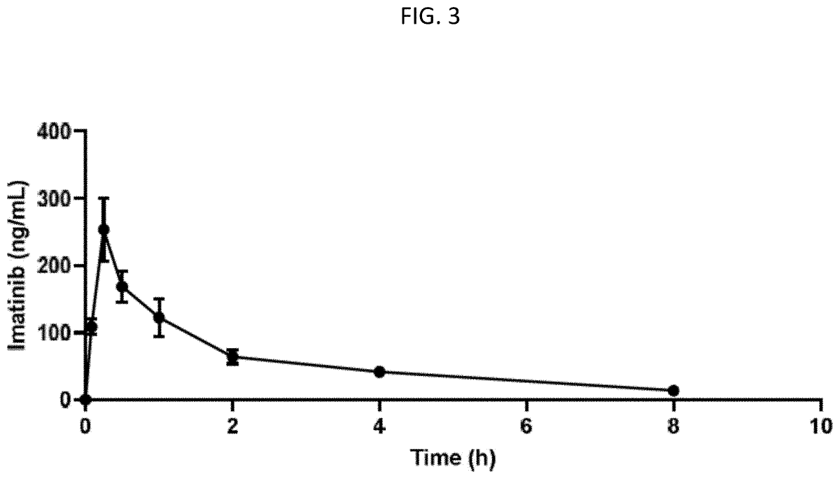

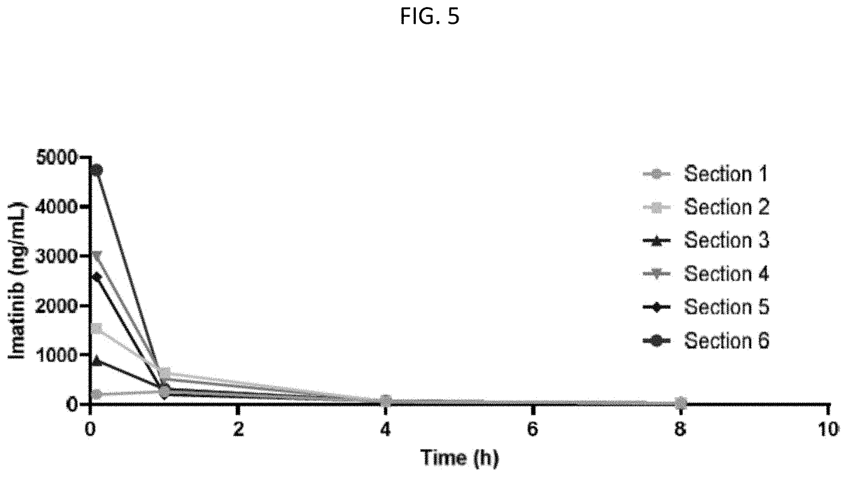

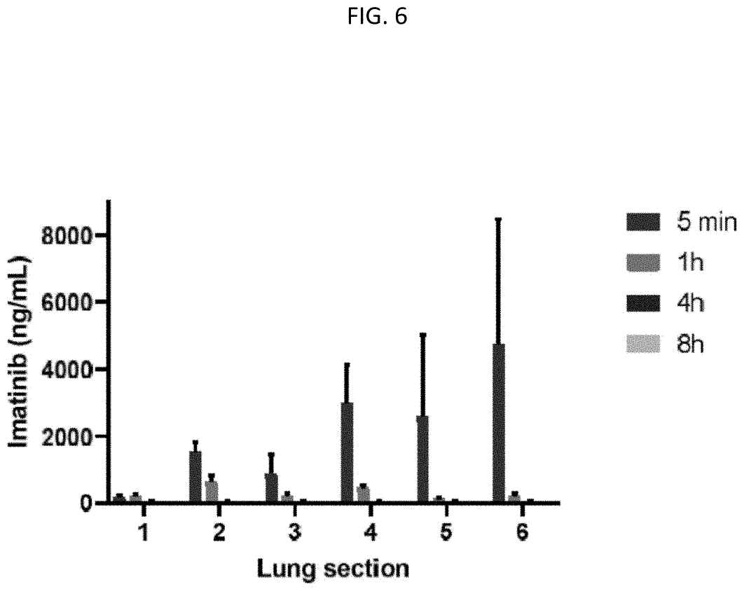

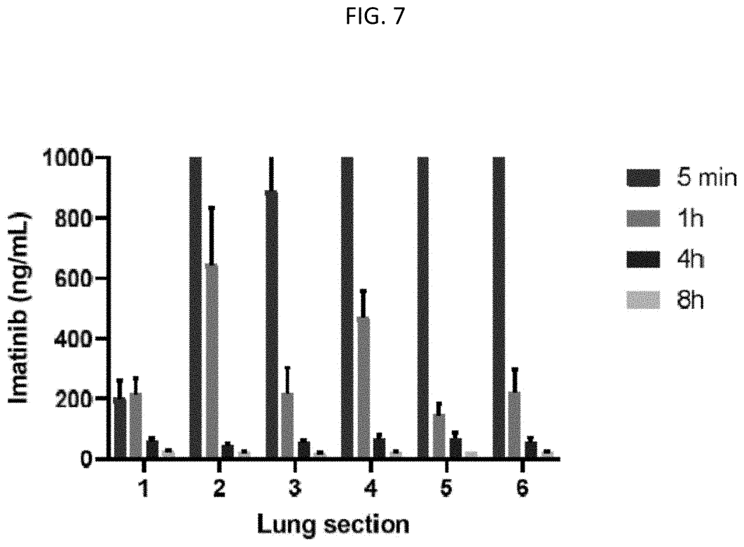

The results are summarized in Table 5 to Table 7 and FIG. 3 to FIG. 7.

TABLE 5
Concentrations of Imatinib in Rat Plasma Samples

| Test | Dose | | Time Point (h) | | | | | | |
|---|---|---|---|---|---|---|---|---|---|
| compound | (mg/kg) | | 0.08 | 0.25 | 0.5 | 1 | 2 | 4 | 8 |
| ImaT | 4 | Average | 108.48 | 253.50 | 168.25 | 136.20 | 64.05 | 41.45 | 13.45 |
| | | SEM | 11.49 | 46.89 | 27.72 | 32.80 | 10.57 | 5.27 | 1.24 |
| | | n | 4 | 4 | 4 | 4 | 4 | 4 | 4 |

TABLE 6
Non-Compartmental Analysis of Plasma After Insufflation of ImaT

| | Elimination | | | |
|---|---|---|---|---|
| Dose | rate | Half-life | Tmax | Cmax |
| mg/kg | L/min | h | h | ng/mL |
| 4 | 3.1E−05 | 2.63 | 0.25 | 253.5 |

TABLE 7
Concentrations of Imatinib in Rat Lung Samples

| | Section 1 | | | Section 2 | | | Section 3 | | | Section 4 | | | Section 5 | | | Section 6 | | |
|---|---|---|---|---|---|---|---|---|---|---|---|---|---|---|---|---|---|---|
| | Lung | | | Lung | | | Lung | | | Lung | | | Lung | | | Lung | | |
| Time | conc | | | conc | | | conc | | | conc | | | conc | | | conc | | |
| point | (ng/ | | | (ng/ | | | (ng/ | | | (ng/ | | | (ng/ | | | (ng/ | | |
| (h) | mL) | sem | n | mL) | sen | n | mL) | sem | n | mL) | sem | n | mL) | sem | n | mL) | sem | n |
| 0.08 | 201.13 | 58 | | 1530.00 | 282 | | 888.50 | 572 | | 2997.50 | 1138 | | 2580.33 | 2450 | | 4737.80 | 3744 | |
| 1 | 260.00 | 88 | 4 | 638.00 | 123 | 4 | 306.75 | 138 | 4 | 510.00 | 64 | 4 | 195.95 | 52 | 4 | 314.05 | 113 | 4 |
| 4 | 62.03 | 8 | | 51.03 | 6 | | 55.93 | 7 | | 68.38 | 14 | | 69.55 | 18 | | 66.28 | 15 | |
| 8 | 27.20 | 2 | | 24.35 | 1 | | 21.15 | 1 | | 24.60 | 1 | | 22.10 | 22 | | 24.60 | 0 | |

This study was conducted to evaluate the pharmacokinetic profile of insufflated ImaT. The delivered dose produced measurable plasma concentrations of Imatinib (See Table 5, FIG. 3). Plasma concentrations of Imatinib were below the limit of detection at the 24 h time point. The characteristic PK profile exhibited tₘₐₓ at the first measured time point (5 min) and terminal declines with t_{1/2} of 2.63 hours (See Table 6). In this study, some rats were excluded from data compilation due to incomplete dosing of those animals. The ImaT powder tended to stick together and formed some aggregates that could not be delivered. These rats were excluded from the dataset prior to analysis.

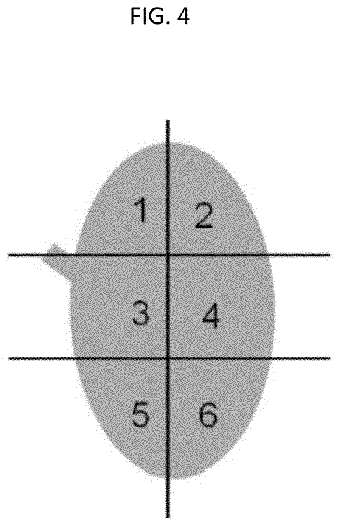

Lungs were also harvested and cut in six sections (See FIG. 4). Each section was homogenized, and Imatinib lung concentration was measured. Lung concentration of Imatinib was maximal at 5 minutes post-inhalation of ImaT. The maximal concentration varied from one lung section to another. Imatinib concentrations were higher in the lower and distal region of the left lung lobe. Imatinib did not stick to the lungs and rapidly transferred to the blood circulation. Imatinib concentrations in the lung were below the limit of detection at the 24 hour time point.

### CONCLUSION

In this study, the pharmacokinetic profile of Imatinib was evaluated over 24 hours. A 1 mg (4 mg/kg) dose of ImaT dry powder was administered via insufflation to rats weighing approximately 250 grams and the delivered Imatinib quickly moved from lungs to blood circulation. Results showed plasma and lung concentrations of Imatinib for up to 8 hours, with Imatinib below the limit of detection at 24 h, and a plasma half-life of 2.63 h.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of treating pulmonary hypertension comprising administering, by inhalation, to a subject in need thereof, a therapeutically effective amount of a composition comprising imatinib or a pharmaceutically acceptable salt thereof.

2. The method of claim 1, wherein the composition is a dry powder composition.

3. The method of claim 2, wherein the dry powder composition further comprises a diketopiperazine.

4. The method of claim 2, wherein the administering is performed using a dry powder inhaler.

5. The method of claim 4, wherein the dry powder inhaler comprises a container comprising from 1 mg to 200 mg of the imatinib or the pharmaceutically acceptable salt thereof.

6. The method of claim 1, wherein a single administering event comprises administering a single dose from 1 mg to 200 mg of the imatinib or the pharmaceutically acceptable salt thereof.

7. The method of claim 6, wherein the single dose is administered in 1-3 breaths.

8. The method of claim 1, wherein the administering comprises from 1 to 3 single administering events.

9. The method of claim 1, wherein the composition has a concentration from about 1 wt. % to about 60 wt. % imatinib or a pharmaceutically acceptable salt thereof of total dry weight.

10. The method of claim 1, wherein the composition comprises imatinib mesylate.

11. The method of claim 1, wherein the subject is a human.

12. A dry powder inhalable composition comprising a therapeutically effective amount of imatinib, or a pharmaceutically acceptable salt thereof, and optionally one or more excipients.

13. The dry powder inhalable composition of claim 12, wherein the composition has a concentration from about 1 wt. % to about 60 wt. % imatinib or a pharmaceutically acceptable salt thereof of total dry weight.

14. The dry powder inhalable composition of claim 12, wherein the composition comprises a particle size between about 0.1 and about 10 μm.

15. The dry powder inhalable composition of claim 12, wherein the one or more excipients comprise from about 0.1 wt. % to about 99 wt. % of a diketopiperazine.

16. The dry powder inhalable composition of claim 15, wherein the diketopiperazine is FDKP.

17. The dry powder inhalable composition of claim 12, comprising imatinib mesylate.

18. A dry powder inhaler comprising the dry powder composition of claim 12.

19. The dry powder inhaler of claim 18 containing from 1 mg to 200 mg of the composition.

20. The dry powder inhaler of claim 18 comprising a container containing a single dose of the dry powder composition.

21. A method of preparing the dry powder inhalable composition of claim 12, comprising adding imatinib to a T suspension or XC suspension.

22. The method of claim 21, wherein the imatinib is added as a solution of imatinib mesylate in water.

23. The method of claim 21, comprising forming the T suspension by crystallizing FDKP in the presence of surfactant or forming the XC suspension by spray drying a suspension of FDKP crystals that are not self-assembled into particles.

24. The method of claim 21, further comprising lyophilizing the T suspension or XC suspension after adding imatinib thereto.
